(19) United States
(12) Patent Application Publication
(10) Pub. No.: US 2011/0158920 A1
(43) Pub. Date: Jun. 30, 2011

(54) METHOD OF PRODUCING A COMPOSITION FROM AN OLEOGEL AND AN AQUEOUS GEL AND THE COMPOSITION

(75) Inventors: Timothy Morley, Edinburgh (GB); Julian Robert Walker Fisher, Edinburgh (GB)

(73) Assignee: ARDANA BIOSCIENCE LIMITED, Edinburgh (GB)

(21) Appl. No.: 12/521,715

(22) PCT Filed: Dec. 31, 2007
(86) PCT No.: PCT/GB07/05023

§ 371 Date: Mar. 10, 2010

(30) Foreign Application Priority Data
Dec. 29, 2006 (GB) 0625995.6
Dec. 31, 2007 (GB) PCT/GB2007/005023

(51) Int. Cl. A61K 8/92; A61K 47/38; A61K 8/73; A61Q 17/04; A61K 31/568; A61K 31/57; A61K 31/085; A61K 31/05; A61K 31/573; A61P 15/18

(52) U.S. Cl. 424/59; 514/781; 514/772.4; 514/178; 514/171; 514/718; 514/731; 514/179

(57) ABSTRACT

A method for producing a bigel, the method comprising: (a) providing an oleogel comprising at least one oily agent gelled with at least one cellulose polymer; (b) providing an aqueous gel comprising at least one component whose viscosity is pH dependent, such as a carbomer; (c) mixing the oleogel and the aqueous gel together to form a bigel; and (d) subsequently adjusting the viscosity of the bigel under high flow and low shear stirring to obtain a bigel of a desired viscosity.

The present invention relates to a method for preparing a gel, and in particular to a method for preparing a stable mixture of an oleogel and an aqueous gel suitable for pharmaceutical, veterinary and cosmetic use.

Numerous ways are known to formulate compositions for pharmaceutical and/or cosmetic uses, each of which has their own advantages and disadvantages.

For example, certain pharmaceutical and cosmetic ingredients are only sparingly soluble in water and, therefore, are optimally formulated in an oily composition rather than an aqueous composition. Oily gels (oleogels) can be made by gelling a synthetic, semisynthetic or natural oil (polar or non-polar) to combine the relatively solid consistency of a gel with the total transparency of an oil. However, oleogels have the drawback of feeling greasy and unpleasant, on account of the presence of the oily compound. This unpleasant feel very often discourages users, particularly in the case of dermatological and cosmetic applications. It is thus desirable, for example, to be able to give a body-care oil a relatively solid consistency but with a fresh and pleasant feel, i.e. a non-greasy feel, while at the same time keeping the beneficial properties of the oil.

One approach that has been used to overcome such disadvantages is to include an oily phase as part of an emulsion. An emulsion can be defined as a preparation of one liquid distributed in small globules throughout the body of a second liquid. The dispersed liquid is known as the discontinuous phase or internal phase, and the dispersion medium is the continuous or external phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. However, as is well known in the art, emulsions are unstable, hence emulsifying agents must be added in order to form a stable emulsion (see, for example, Remington: The Science and Practice of Pharmacy, 19^{th} Edition (1995), ISBN: 0-912734-04-3, Chapter 21, pages 282-291).

A further formulation approach is described in EP 1 083 880 B1 which relates to the formation of a stable mixture of certain types of oleogels and an aqueous gel, without an emulsifying agent. Such a stable mixture is also known as a “bigel”. The entire disclosure of EP 1 083 880 B1 relating to the formation of a bigel is incorporated herein by reference.

It is commonly accepted practice in the formulation industry to optimise the components of formulations to the desired pH and viscosity before mixing them together. This is both more convenient and minimises the financial loss caused by a bad batch of any particular component. For example, in the field of emulsions, once an emulsion of the desired viscosity in the form of a cream or lotion has been made, each additional formulation step increases the risk of breaking the emulsion into its constituents. Therefore, it is generally considered preferable to optimise the individual components prior to forming the final composition.

It is also commonly accepted practice in the formulation industry to use high shear mixing/stirring when seeking to obtain a homogenous viscous product. This is particularly true in the case of creams and ointments (see, Silverson® Application Reports 18PA2 “Solutions for your toughest mixing applications in pharmaceuticals: Production of pharmaceutical creams and ointments”, and 5TA2 “Solutions for your toughest mixing applications in cosmetics: Production of cosmetic creams and lotions”).

The present inventors have found that, particularly for pilot- and large-scale formulation of bigel creams and ointments, it is advantageous not to optimise the pH and viscosity of the aqueous phase to its final value (i.e., neutralisation) prior to mixing the aqueous phase with the oily phase. On the contrary, it is advantageous to optimise the pH of the aqueous phase components after blending with the oily phase. This results in the thickening of the bigel only after the two phases have been mixed together, which allows a bigel cream or ointment of the desired viscosity to be attained.

Moreover, the present inventors have determined that the commonly-accepted practice of using high shear mixing is not appropriate for producing a bigel. The inventors have shown that if high shear mixing alone is used during the neutralisation stage the gel matrix is broken down, resulting in a reduction of the product viscosity, and the bigel begins to split. Also, the inventors have shown that if the rate of mixing is too low, there is incomplete incorporation of the neutralising agent resulting in the splitting of the bigel.

Surprisingly and unexpectedly, the inventors have shown that the combination of high flow and low shear mixing conditions is required to obtain a bigel cream or ointment, at least for commercial scale production.

A first aspect of the invention thus provides a method of producing a bigel, the method comprising:
(a) providing an oleogel comprising at least one oily agent gelled with at least one cellulose polymer;
(b) providing an aqueous gel comprising at least one component whose viscosity is pH dependent;
(c) mixing the oleogel and the aqueous gel together to form a bigel; and
(d) subsequently adjusting the viscosity of the bigel under high flow and low-shear mixing to obtain a bigel of a desired viscosity.

As used herein, the terms “mixing” and “stirring” are used interchangeably.

By a “bigel” we mean a stable mixture of substantially uniform appearance to the naked eye which is an intimate mixture of the oleogel and the aqueous gel, and not an emulsion. In other words, the bigel is a uniform dispersion of one in the other, such that only a single gel can be distinguished when inspected visually.

Further, no separation of the aqueous gel and oleogel phases is detected when a bigel is applied to the skin.

By “stable” we include the meaning that the bigel retains a substantially uniform appearance without any demixing of the oily and aqueous gels on storage at room temperature for a period of up to two months, and preferably for up to six, or even twelve months.

A bigel is not an emulsion, and there are a number of structural differences between a bigel and a both a water-in-oil and an oil-in-water emulsion which causes a bigel to have significantly improved properties as a pharmaceutical or cosmetic composition. For example, in a bigel the physico-chemical stabilisation process is an actual entrapment via a three dimensional gel network. This results in an extra-fine dispersion which can be viewed microscopically. Also, because of the absence of a emulsifier, i.e. the bigel can be emulsifier-free, a bigel does not have an interfacial film. As a result the interface between the continuous and non-continuous phases of the bigel are conveniently not stabilized by an emulsifier. This provides significant advantages compared to traditional emulsions, e.g. a natural water resistance and an improved ability to formulate incompatible ingredients, such as hydrophilic and lipophilic active ingredients, in the same product.

The intimate nature of the mixture of the aqueous and oily gels in a bigel can be observed, for example, by introducing a dye substance into either the oily (e.g. Soudan red 3) or aqueous (e.g. erythrosine) gel phases before forming the bigel. On visual inspection the dye is seen to be uniformly dispersed, even though the dye is present in only one of the oleogel or aqueous gel. Indeed, if two different dyes of different colours are introduced into the oily and aqueous phases, respectively, before forming the bigel, both colours can be observed uniformly dispersed throughout the bigel. This is in contrast to an emulsion wherein if a dye that is either water-soluble or oil-soluble is introduced into the aqueous or oily phases, respectively, before forming an emulsion, only the colour of the dye in the external phase will be observed (Remington: The Science and Practice of Pharmacy, 19^{th} Edition (1995) Chapter 21, page 282).

A bigel can also be distinguished from an emulsion by virtue of a different pattern of electrical conductance. Electricity can be conducted across an oil-in-water emulsion. This can be seen by using an electrical conductance measuring system (e.g. Typomat, SKM Electronik, Germany, or which can be made using standard laboratory equipment), made of two electrodes at a separation of e.g. 1 cm, between which the emulsion is placed for conducting the electricity, and the conductance can be observed using e.g. a coloured light as a readout. In the case of a water-in-oil emulsion, there is no electrical conductance, and no coloured light, irrespective of the relative concentrations of the aqueous and oily phases. In the case of a bigel, whatever the percentage of the aqueous and oily phases, electrical conductance is always visible because the particles of water, despite being separated, are in touch with each other and allow the electricity to cross.

Since a bigel is not an emulsion it does not need to contain an emulsifying agent such as a surfactant to stabilise it, and is stable in the absence of an emulsifier. Thus, preferably, in the preparation of a bigel, an emulsifying agent is not added. Typical emulsifying agents that do not need to be present in the bigel include: anionic surfactants selected from alkylaryl sulphonates such as sodium dodecylbenzene sulphonate, calcium alkylbenzene sulphonate, triethanolamine alkylbenzene sulphonate, monoisopropylamine dodecylbenzene sulphonate, sodium diisopropylnaphthalene sulphonate, and calcium alkyl aryl sulphonate; alcohol sulphates such as sodium lauryl sulphate, lithium lauryl sulphate, triethanolamine lauryl sulphate, sodium ethylhexyl sulphate, monoethanolamine lauryl sulphate, and sodium tetradecyl sulphate; ether sulphates such as sodium lauryl ether (2EO) sulphate, ammonium alkyl ether sulphate, ammonium lauryl ether (3EO) sulphate, and sodium alkyl lauryl ether sulphate; phosphate esters such as diethanolamine cetyl phosphate, sodium lauryl ether phosphate, acid alkyl phosphoric ester, and triethanolamine complex phosphate ester; sulphosuccinates such as sodium sulphosuccinate and sodium dioctyl sulphosuccinate; sarcosinates such as sodium lauroyl sarcosinate and ammonium lauroyl sarcosinate; and other anionic surfactants such as paraffin, olefin and petroleum sulphates/sulphonates, taurates and isethionates, and carboxylates; quaternary ammonium cationics such as distearyl dimethyl ammonium chloride, lauryl trimethyl ammonium chloride, cetyl trimethyl ammonium chloride, alkyl dimethyl benzyl ammonium chloride, alkyl trimethyl ammonium methosulphate, coco trimethyl ammonium chloride, cetyl pyridinium chloride, and didecyl dimethyl ammonium chloride; non-ionic surfactants selected from alkyl phenol ethoxylates such as nonyl phenol ethoxylate (9EO), nonyl phenol ethoxylate (2EO), and octyl phenol ethoxylate (10EO); alcohol ethoxylates such as C₁₂/C₁₄ synthetic ethoxylate (8EO), stearyl alcohol ethoxylate (7EO) and cetostearyl alcohol ethoxylate (20EO); amine ethoxylates such as coconut fatty amine ethoxylate (10EO); ester ethoxylates such as sorbitan monolaurate ethoxylate; EO/PO block polymers such as 80% PO/20% EO; alkanolamides such as coconut diethanolamide; and esters such as sorbitan monolaurate, sorbitan monolaurate 4EO, di-isopropyl adipate and cetostearyl stearate; and amphoteric surfactants such as coco imidazoline betaine, coco amido sulpho betaine, oleo amido propyl betaine and tall oil imidazoline.

It is, however, also appreciated that certain of the above emulsifying agents may be included in a bigel as absorption promoters for drugs, and not as emulsifying agents per se, and if included they are typically present at concentration below that needed to stabilize an emulsion Emulsifying agents that can act as an absorption promoters include Tweens, Spans, alkylaryl sulphonates, SDS, and DOSS.

Preferably, the method is for pilot- or large-scale manufacture of a bigel. By “pilot-scale” we mean batches of 5-100 kg, including batches of 5-10 kg, 10-50 kg, or 50-100 kg. By “large-scale” we mean of batches of over 100 kg, such as 100-200 kg, or 200-400 kg, or 400-1,000 kg, or more. The method described in the Examples below has been validated for a batch of 40 kg. According to industry standards this is sufficient validation for batches up to 400 kg.

As mentioned above, the oleogel (also referred to as oily phase or oily gel) comprises an oily agent gelled with at least one cellulose polymer. The cellulose polymer may be chosen from ethylcellulose, non-sodium carboxymethylcellulose, and mixtures thereof. Ethylcellulose is preferred, and is commercially available as Aqualon® Ethylcellulose (Hercules) and as Ethocel™ (Dow Chemical Company).

The oily agent may preferably be chosen in particular from mono-, di-, and triglycerides of synthetic, semi-synthetic and natural origin, and mixtures thereof. Synthetic mono-, di- or triglycerides may include Miglyol 810 and 812 (Dynamit Nobel). As semisynthetic mono-, di- or triglycerides, reference is made in particular to propylene glycol isostearate, such as the product sold under the name “hydrophilol isostearique” (Gattefossé), and the polyglycolysed glyceride “Labrafil® M 1944 CS” (Gattefossé). Labrafil® M 1944 CS is a mixture of polyoxy ethylenated oleic glycerides obtained by alcoholysis of natural plant oil (French Pharmacopoeia, 8th edition). It is an oily liquid whose properties include a liquid drop point; a saponification number of 145/175; an acid number of <2; an iodine number of 60/90; an oral lethal dose (OLD) in the rat of >20 ml/kg; and a hydrophile/lipophile balance (HLB) of 3/4.

Lastly, as mono-, di- or triglycerides of natural origin, reference is made in particular to oils of plant origin, such as sweet almond oil, argan oil and palm oil.

The oily agent may also comprise a mixture of capric/caprylic triglycerides such as Labrafac CC® (Gattefossé).

In an alternative embodiment, the oily agent may be chosen from the oily sunscreen filters and more preferably from the cinnamic acid esters. In particular, such an oily agent may be chosen from the group consisting of 4-methoxycinnamic acid isopentyl ester, 4-methoxycinnamic ethyl 2-hexyl ester and mixtures thereof. Preferably, the sunscreen filter as the oily agent may be 4-methoxycinnamic acid ethyl 2-hexyl ester, such as the product sold under the name “Parsol MCX” (Roche). The oily sunscreen filter may be present in a proportion of about 0.1 to about 20% by weight, and preferably about 4 to about 10% by weight, relative to the total weight of the bigel produced according to the invention. The bigel thus produced may be used as a sunscreen filter composition, optionally comprising a further active ingredient such a pharmaceutical or cosmetic active ingredient, as below described.

In an embodiment, ethylcellulose is present in a proportion of between about 0.01 and 5% by weight of the bigel produced, more preferably between about 0.05 and 1% by weight. In some embodiments, the amount of ethylcellulose is a low percentage of about 0.05-0.1%, while in other embodiments it can be higher, such as 0.1-0.5%, 0.5-1% or 1-5% by weight of the bigel produced.

Typically, the ethylcellulose is present in a proportion of between about 0.1 and 10% by weight of the oily phase, more preferably between about 0.5 and 5% by weight. In some embodiments, the amount of ethylcellulose is a low percentage of about 0.5-1%, while in other embodiments it can be higher, such as 1-3% or 3-5% by weight of the oleogel.

As is well known in the art, the level of gelling provided by ethylcellulose in the oleogel, and hence its viscosity, depends upon both the proportion and the grade of the ethylcellulose. For a given amount by weight of ethylcellulose in an oleogel, the higher the grade of ethylcellulose used, the higher the viscosity of the oleogel. In other words, higher amounts of ethylcellulose will generally be used when the grade of the ethylcellulose is lower. For the Dow Ethocel® ethylcelluloses, information on the relationship between viscosity, concentration and grade of product is available in “Ethocel, Ethylcellulose Polymers Technical Handbook” (http://www.dow.com/ethocel/resource/lit.htm). For the Aqualon® ethylcelluloses, information on the relationship between viscosity, concentration and grade of product is available in the Aqualon® Product Data Sheet (250-49A 7-03) and in “Aqualon® Ethylcellulose (EC) Physical and Chemical Properties” (Aqualon® Product Booklet 250-42A), both of which are available at www.herc.com/aqualon. Each of these references relating to the properties of ethylcellulose is incorporated herein by reference.

According to one specific embodiment, the oily phase comprises ethylcellulose in a proportion of between about 1 and about 10% by weight, and the oily agent comprises a mixture of polyoxy ethylenated oleic glycerides in a proportion of between about 5 and about 90% by weight, relative to the total weight of the oleogel.

According to another specific embodiment, the oily phase comprises ethylcellulose in a proportion of between about 1 and about 10% by weight, and the oily agent comprises a mixture of capric/caprylic triglycerides in a proportion of between about 5 and about 90% by weight, relative to the total weight of the oleogel.

It is preferred if the oleogel further comprises propylene glycol isostearate. The propylene glycol isostearate is typically present in a proportion of between about 0.1 and about 5% by weight, relative to the total weight of the oleogel. It is preferred if the propylene glycol isostearate is present in a proportion of between about 0.05 and about 1% by weight, relative to the total weight of the bigel produced. In some embodiments, the amount of propylene glycol isostearate is a low percentage of about 0.05-0.5%, while in other embodiments it can be higher, such as 0.5-1% by weight. Propylene glycol isostearate is commercially available under the following trade names from the listed suppliers: Emerest 2384 (Henkel/COSPHA); Hydrophilol isostearique (Gattefossé s.a); Prisorine PMIS 2034 (Unichema); and Witconol 2384 (Witco), and from A & E Connock Ltd, Fordingbridge UK.

It is also preferred if the oleogel further comprises propylene glycol laurate. The propylene glycol laurate is typically present in a proportion of between about 1 and about 50% by weight, relative to the total weight of the oleogel. It is preferred if the propylene glycol laurate is present in a proportion of between about 1% and about 10% by weight, relative to the total weight of the bigel produced. In some embodiments, the amount of propylene glycol laurate is a low percentage of about 1-5%, while in other embodiments it can be higher, such as 5-10% by weight. Propylene glycol laurate is commercially available under the following trade names from the listed suppliers: Calgene PGML (Calgene); Imwitor 412 (Huls AG/Huls America); Jeechem PGML (Jeen); Laurate De Propylene Glycol (Prod'Hyg); Schercemol PGML (Specialty Industrial); and Unipeg-PGML (Universal Preserv-A-Chem), and from A & E Connock Ltd, Fordingbridge UK. It is appreciated that commercially obtainable preparations of propylene glycol laurate are often blends of the laurate monoester and the diester. Reference herein to amounts or proportions of propylene glycol laurate include both the laurate monoester and diester forms.

More preferably, the oleogel further comprises both propylene glycol isostearate and propylene glycol laurate.

Most preferably, the oleogel comprises ethylcellulose, propylene glycol isostearate and propylene glycol laurate and at least one oily agent.

Preferably, the ethylcellulose, propylene glycol isostearate and propylene glycol laurate are present, in total, in a proportion of between about 1% and 10% by weight of the bigel composition, more preferably between 2% and 6%. In preferred embodiments, the ethylcellulose, propylene glycol isostearate and propylene glycol laurate are present, in total, at about 3% or 4% or 5% or 6% by weight of the bigel composition.

In a preferred embodiment of the present invention, the ethylcellulose, propylene glycol isostearate and propylene glycol laurate are present, in relative proportions of about 2-10%:2-10%:80-96%, respectively. For Grade 50 ethylcelluloses, the proportion of ethylcellulose is preferably between 3-6%, and more preferably between 4-5%, and preferred relative proportions are 4-5%:5-6%:89-91%, respectively. For other grade ethylcelluloses, the proportion of ethylcellulose can be calculated accordingly. In one specific embodiment using a lower grade ethylcellulose, the ethylcellulose, propylene glycol isostearate and propylene glycol laurate are present, in relative proportions of about 8:2:90%, respectively.

A suitable source of the ethylcellulose, propylene glycol isostearate and propylene glycol laurate is Emulfree® P (Gattefossé).

In a preferred embodiment, the oily phase comprises ethylcellulose, propylene glycol isostearate and propylene glycol laurate, and a mixture of capric/caprylic triglycerides as an oily agent, optionally with a further oily agent(s). Typically, the ethylcellulose, propylene glycol isostearate and propylene glycol laurate, when taken together, are present at about 1-10% by weight of the final bigel, preferably between 1-9%, 2-8%, or 2-7%, and most preferably at about 3%, 4% or 5% or 6% by weight of the final bigel. Also preferably, the ethylcellulose, propylene glycol isostearate and propylene glycol laurate are present, in the relative proportions discussed above. Also, typically, the mixture of capric/caprylic triglycerides is present at between 10-30% by weight of the final bigel, preferably between 15-25%, and more preferably at about 20% by weight of the final bigel.

In an embodiment, step (a) of providing an oleogel may comprise making an oleogel. The manufacture of an oleogel is well known to a person of skill in the art, and the oleogel is typically made using the components described above. A suitable method for making an oleogel is also described in the Examples.

The preparation of suitable oily gels is also described in EP 0 356 325 B1, the relevant parts of which are incorporated herein by reference.

In an embodiment, the oily phase can be made by combining ethylcellulose, propylene glycol isostearate and propylene glycol laurate at 120-150° C. under nitrogen with constant stirring. Preferably, the ethylcellulose, propylene glycol isostearate and propylene glycol laurate are combined, in the relative proportions discussed above. After stirring, the mixture is allowed to cool to room temperature and is combined with an oily agent, such as a mixture of capric/caprylic triglycerides, as described in the examples.

In certain preferred embodiments, the oily phase is in the form of a viscous liquid rather than a gel. However, since the viscosity of the oily phase is difficult to evaluate with conventional systems (cylinders or cone-plate) due to the gliding effect of the oils, the actual viscosity of the oily phase is not usually measured. Nevertheless, as is understood in the art, certain variations in the viscosity of an oily phase can be determined visually or by feel.

The method may also comprise adjusting the viscosity of the oily phase prior to mixing it with the aqueous phase in step (c), where necessary. Typically, the viscosity adjustment is performed by heating the oily phase preferably to a temperature of not more than 50° C., although other suitable methods may be employed as are well known in the art.

For liquids and solutions where the mechanism of fluid flow is simple the viscosity may be accurately determined by means of an Ostwald viscometer in which the liquid flows down through a glass capillary tube and is timed between two points. There are many variations of the capillary flow method among which the flow cup is a quick and simple method for used in the mixing plant.

For liquids that do not have simple flow characteristics, viscometers based upon rotational methods, rotating cylinders or plates, are used. Here the liquid placed between one or more rotating surfaces is subjected to shearing forces and its resistance to them is measured. Further useful information is obtained by studying how the liquid under test behaves with different shearing rates. This is particularly valuable in the development of preparations that are to be subjected to mechanical action in use or during dispensing from their container. For example non-drip emulsion paints are gels that reduce in viscosity when put under a shearing force created by the paint brush moving against the gel surface. With such preparations it is essential to study flow characteristics far beyond simple viscosity determinations. A widely-used rotational method for measuring viscosity is the Brookfield viscometer. This instrument enables shearing to be carried out continuously and so allows the study of different conditions and at different shearing rates. Viscosities of suspensions, pastes and gels are commonly determined by means of the Brookfield viscometer (with mobiles) or a Rheomat viscometer (coaxial cylinders).

A further method for measuring viscosity is to use a Lamy VRM-08 viscometer (which is equivalent to a Rheomat R180). The Lamy VRM-08 is used in conjunction with an MS DIN 1.3 module when measuring more viscous compositions, including those over 100 Pa·s., while an MS DIN 1.9 module is used with less viscous compositions, and can measure viscosities as low as 3 mPa·s. Unless mentioned otherwise, all viscosity measurements in the context of this invention are taken using a Lamy VRM-08 with the appropriate MS DIN module at 23° C. A shear stress of 0.8 s⁻¹ is typically used, with a measurement taken after 30 seconds.

The aqueous gel (also known as the aqueous phase) comprises at least one component whose viscosity is adjusted in the process according to the invention. In a preferred embodiment, the viscosity is adjusted by adjusting the pH of the bigel under high flow and low shear mixing to obtain a bigel of the desired viscosity. However, other methods of adjusting the viscosity are known to those skilled in the art, and include the use of thickening agents whose viscosity may be adjusted by other factors such as changing the salt or ion concentration. Such thickeners whose viscosity is ion or salt (e.g. sodium chloride) dependent are known to the skilled person.

Preferably, to adjust the viscosity the composition comprises at least one component whose viscosity is pH dependent, which act as gelling agents when the pH is suitably adjusted. Suitable components include Carbopols®, now known as carbomers, and sodium carboxymethylcellulose. Carbomers are preferred.

Carbomer is a generic name for a family of polymers known as Carbopols®. A carbomer is a homopolymer of acrylic acid cross-linked with an allyl ether of pentaerythritol, an allyl ether of sucrose, or an allyl ether of propylene. As a group, they are dry powders with high bulk densities, and form acidic aqueous solutions (pH around 3.0), which thicken at higher pHs (around 5 or 6). They swell in aqueous solution of that pH as much as 1000 times their original volume, and their solutions can range in viscosity from 0 to 80,000 centipoise (cP). Some examples of carbomers are listed in Table 1.

TABLE 1

| Polymer Name | Viscosity cP * |
|---|---|
| Carbopol ® Ultrez | 45,000-65,000 |
| 10 NF | |
| Carbopol ® 71G NF | 4,000-11,000 |
| Carbopol ® 910 | 3,000-7,000 |
| Carbopol ® 934 NF | 2,050-5,450 (0.2%). 30,500-39,400 (0.5%) |
| Carbopol ® 934P NF | 2,050-5,450 (0.2%). 29,400-39,400 (0.5%) |
| Carbopol ® 940 NF | 40,000-60,000 |
| Carbopol ® 941 NF | 4,000-10,000 |
| Carbopol ® 971P NF | 4,000-11,000 |
| Carbopol ® 974P NF | 29,400-39,400 |
| Carbopol ® 980 NF | 13,000-30,000 (0.2%). 40,000-60,000 (0.5%) |
| Carbopol ® 981 NF | 1,000-6,000 (0.2%). 4,000-10,000 (0.5%) |
| Carbopol ® 1342 NF | 9,500-26,500 (1.0%). |
| | 5,500-15,000 (1.0% dispersion with 1.0% salt) |

* The viscosities listed in Table 1 were taken from the Product Specifications for the Noveon ™ Carbopol ® polymers, incorporated herein by reference (http://www.pharma.noveoninc.com/literature/specs/specsCarbopol.asp), each measured using a Brookfield viscometer at 20 rpm at 25° C., using a neutralised 0.5% dispersion except where indicated otherwise.

Sodium carboxymethylcellulose (CMC) maintains its viscosity at pH 7-9; however the viscosity dramatically decreases below pH 4 and above pH 10, and so, at least within these pH transitions, is also considered to be a component whose viscosity is pH dependent. However, CMC is less preferred than the carbomers, and typically is used when a desired Active Pharmaceutical Ingredient (API) is incompatible with the carbomer. The relationship between the concentration and viscosity of a composition containing carboxymethylcellulose are known to the skilled person (see, for example, Hercules, Inc. Technical Bulletin VC-453C (2001) “Rheology of Aqualon® Water-Soluble Polymers in Solution”, especially FIG. 4 therein (http://www.herc.com/aqualon/pharm/index.html), incorporated herein by reference).

Other gelling agents that may be included in the aqueous phase are preferably chosen from poloxamers)(Pluronics®, acacia, alginic acid, bentonite, ethylcellulose, hydroxyethyl cellulose, hydroxypropyl cellulose, gelatin magnesium aluminum silicate (Veegum®), methylcellulose, polyvinyl alcohol, sodium alginate, tragacanth, and xanthan gum, and mixtures thereof.

With the exception of carboxymethylcellulose, the cellulose derivatives (such as methylcellulose, hydroxyethylcellulose, hydroxypropylcellulose, and hydroxypropylmethylcellulose) maintain their viscosity over a wide pH range (3-11), and are not considered to be components whose viscosity is pH dependent.

Poloxamers (Pluronics®) are copolymers of polyoxyethylene and polyoxypropylene. They form thermoreversible gels in concentration ranging from 15% to 50%. This means that they are liquids at cool (refrigerator) temperature, but are gels at room or body temperature. Poloxamer copolymers are white, waxy granules that form clear liquids when dispersed in cold water or cooled to 0-10° C. overnight. In certain embodiments, the property of gelling at increased temperatures may be utilised in obtaining an aqueous gel of the desired viscosity for mixing with the oily gel, before increasing the pH and hence the viscosity of the carbomer to obtain the final desired viscosity of the bigel.

It is appreciated that Carbopol® 934P, methylcellulose, hydroxypropyl-methylcellulose, and sodium carboxymethylcellulose are preferred for oral administration.

Typically, the gelling agent whose viscosity varies and conveniently is pH dependent is present in a proportion of between about 0.1 and about 30% by weight of the aqueous gel, preferably between 0.5 and 25%, or between around 0.5% and 20%, depending upon the desired viscosity and other properties of the bigel to be produced.

Preferably, the gelling agent whose viscosity varies and conveniently is pH dependent is present in a proportion of between about 0.3 and about 5% by weight of the bigel, for example at about any of the following proportions: 0.4%, 0.5%, 0.6%, 0.7%, 0.8%, 0.9%, 1.0%, 1.1%, 1.2%, 1:3%, 1.4%, 1.5%, 1.6%, 1.7%, 1.8%, 1.9%, 2.0%, 2.1%, 2.2%, 2.3%, 2.4%, 2.5%, by weight, or between 2.5-3%, 3-3.5%, 3.5-4%, 4-4.5% or between 4.5-5% by weight of the bigel produced.

More particularly, the gelling agent whose viscosity is pH dependent for the aqueous gel may be the carbomer Carbopol 974 or Carbopol 980 NF, present in a proportion of between about 0.1 and about 5% by weight, and preferably between 0.4 and 2% by weight relative to the total weight of the bigel produced.

In an embodiment, step (b) of providing an aqueous gel may comprise making an aqueous gel. The manufacture of an aqueous gel is well known to a person of skill in the art, and the aqueous gel is typically made using the components described above. A suitable method for making an aqueous gel is described below in the Examples. Typically, the aqueous phase is formed by mixing using a shear mixer.

The method may also comprise measuring the viscosity of the aqueous phase prior to mixing it with the oily phase in step (c).

The present inventors have shown that, at least for pilot- or large-scale preparations of a bigel, if the aqueous gel phase is too viscous, the oily phase is not incorporated properly when the phases are mixed together in step (c), and an optimal bigel is not obtained. Similarly, if the aqueous gel phase is not viscous enough, the oily and aqueous phases are not incorporated properly when they are mixed together, and an optimal bigel is not obtained.

In other words, in the pilot- or large-scale formation of a bigel there is an optimum viscosity range for the aqueous phase at the stage when it is mixed together with the oily phase. (There may also be a preferred viscosity range for the oily phase at the stage when it is mixed together with the aqueous phase, although this appears to be less critical.) Preferred viscosities of the aqueous phase, prior to mixing with the oily phase in step (c), range between about 100 and about 1,000 mPa·s (cP) (measured using a Lamy RM-08 with an MS Din 1.9 module at 23° C. under a shear stress of 0.8 s⁻¹).

The method may also comprise adjusting the viscosity of the aqueous phase prior to mixing it with the oil phase in step (c), where necessary. Typically, the viscosity adjustment is performed by heating the aqueous phase preferably to temperature of not more than 50° C., although other suitable methods may be employed as are well known in the art.

In an embodiment, in order to obtain a preferred viscosity for mixing purposes, it is possible to partially pre-neutralise the aqueous phase before it is mixed with the oily phase in step (c). However, for the avoidance of doubt, the method of the present invention still requires a further step (d) of adjusting the pH of the bigel using high flow and low shear stirring to obtain a bigel of a desired viscosity.

The ratio of the weight of oleogel to the weight of aqueous gel may be between about 10:90 and about 90:10, preferably between 30:70 and 70:30, and more preferably between 40:60 and 60:40. The actual ratio chosen typically depends upon the active ingredients(s) present in each phase, particularly since it is appreciated that there is a higher bioavailability from the aqueous phase, as well as upon the desired physical characteristics of the bigel to be achieved. For example, the higher the percentage of oil, the higher the oily feel to the bigel.

The relative proportion of aqueous gel is typically such that the final proportion of carbomer used is between 0.2 and 2%, and more preferably between 0.5-1.5%, by weight, of the final bigel composition: In a specific embodiment, the amount of carbomer in the final bigel composition is around 1.4-1.5%.

Step (c) of mixing the oleogel and aqueous gel together typically comprises incorporating the oily phase into the aqueous phase until a mixture of substantially uniform appearance is obtained. In other words, step (c) typically comprises producing a homogenous bigel of low viscosity and substantially uniform appearance. Alternatively, the aqueous phase can be incorporated into the oily phase until a homogenous mixture of substantially uniform appearance is obtained.

Particularly when the aqueous gel comprises a carbomer, the oleogel and aqueous gel are added together and mixed at high speed, such as 1600-3000 rpm, typically over a period of around 15-25 minutes. Optionally, the mixing can be maintained for a further 15-25 minutes after the addition has been completed, preferably under a vacuum. Since at this stage the mixture has not been neutralised, the polymeric carbomer gel matrix has not yet fully formed, hence the high speed mixing does not destroy the polymeric gel matrix. Suitably, a high velocity turbine mixer, such as a Trimix® type mixer (Rayneri) may be used. Alternatively, as described in the Examples, incorporation of the oily phase into the aqueous phase can be carried out using a 50 L OLSA MPEF with a rotor/stator type disperser homogeniser at 3000 rpm in conjunction with a planetary mixer at a medium speed (20 to 35 rpm).

The other operating conditions for carrying out the present process are well within the ordinary scope of those skilled in the art.

As mentioned above, the inventors have realised that if the aqueous gel is too thick the oily phase is not incorporated optimally when the two phases are mixed together on a large scale, and an optimal bigel is not obtained. Since adjusting the pH of the aqueous gel makes it become more viscous, for pilot- and large-scale formulation of bigels, the pH adjustment is conducted after mixing the aqueous gel with the oleogel which results in the thickening of the bigel to the desired level. Thus, typically, step (d) of adjusting the pH of the bigel under high flow and low-shear stirring/mixing raises the viscosity of the bigel made in step (c) until a desired viscosity is attained. To this end, the low shear step (d) may ideally comprise a step carried out at sufficiently low shear to provide a bigel of desired viscosity. If step (d) is carried out with mixing at too high shear, the resultant bigel may have a viscosity which is too low, due to excessive disruption of the matrix formed by the components whose viscosity is pH dependent.

It is appreciated that step (d) of adjusting the pH of the bigel under high flow and low-shear stirring to obtain a bigel of a desired viscosity, may be considered to be a neutralisation step, particularly when the pH of a relatively acidic mixture is increased towards neutral pH.

High flow stirring or mixing is well known in the art, and the skilled person is well aware of the differences between high flow and low flow stirring. For example, the IKA® technical publication “Mixing and Processing Technology”, 2003, describes a number of mixing devices and provides details of their nominal flow rates for various sizes of mixers (http://www.ikausa.com/pdfs/2003_process_Catlog2.pdf). The Ultra Turrax™ UTL 2000/ . . . 2P series is described as a high flow mixing tool and is stated to have a nominal flow rate of 1500 gpm/340,000 l/h (+25% depending on fluid properties and auxiliary pumping) for the UTL 2000/50 2P which has the largest volume. (In this instance, gpm means US gallons per minute). The other mixers described in this IKA® technical publication have much lower flow rates.

Having a high flow rate while adding the neutralisation agent spreads the agent out rapidly and consistently through the bigel, so that a mixture of homogenous pH is obtained as quickly as possible.

By “low-shear” stirring or mixing we include non-shear stirring or mixing. Low-shear stirring is well known in the art, and the skilled person is well aware of the differences between high-shear and low-shear (including non-shear) stirring. By low-shear stirring we mean a non-destructive process of mixing the bigel without significantly breaking up the gel-matrix of the bigel.

Thus, step (d) of adjusting the pH of the bigel under high flow and low shear mixing to obtain a bigel of a desired viscosity, may be considered to include rapidly adjusting the pH of the mixture under mixing conditions that provide a homogenous mixture without breaking up the gel-matrix of the bigel, i.e. under mixing conditions that do not significantly break up the polymeric chains of the gelling agent in the bigel. See, Encyclopaedia of Pharmaceutical Technology 2^{nd} Edition, 2002, Eds. Swarbrick & Boylan, Marcel Dekker, ISBN 0-8247-2825-4 and Remington: The Science and Practice of Pharmacy 19^{st} Edition, 1995, Chapter 86, pages 1495-1523, for further information on high flow and low shear stirring.

In an embodiment, the desired high flow and low shear mixing conditions can be obtained using a combination of mixers. For example, a low flow and low shear planetary or gate mixer, such as those sold by Hobart or Kenwood, set to a maximum speed of about 60 rpm can be used in combination with a high shear mixer with medium to high flow, such as a Silverson type mixer, set to a speed of about 1500 rpm. The planetary mixer is typically used at a speed from 10 to 60 rpm, and more preferably from 30 to 55 rpm, while the Silverson type mixer is typically used at its low speed setting of about 1500 rpm (+/−20%). With the additional flow generated by the planetary mixer in combination with the high shear mixer, the neutralising agent can be incorporated into the gel without too much degradation of the carbopol gel matrix. This can result in a gel with a viscosity greater than 300,000 cP (using the method of viscosity measurement specified in the Examples) and a consistent stable gel matrix.

As shown in Tables 4 and 5 in Example 1, addition of the Na methylparaben raised the pH of the mixture to about 3.9 which resulted in a considerable increase in viscosity. Further neutralisation with NaOH to about pH 4.95-5.0 under high flow and low shear mixing conditions over a total time period of 55 minutes resulted in a 30-40% reduction in viscosity of the bigel produced. Accordingly, by high flow and low shear mixing we include conditions that result in no more than a 30-40% reduction in viscosity when neutralising with NaOH from pH 3.9 to pH 4.9 over a time period of 55 minutes. Typically, conditions that result in a reduction in viscosity of at least 40%, or at least 50%, or more, when neutralising with NaOH from pH 3.9 to pH 4.9 over a time period of 55 minutes are not high flow and low shear mixing.

Thus, by “high flow and low shear mixing” we include the meaning of mixing under flow and shear conditions which are equivalent to those obtained using the combination of a planetary mixer and a high shear mixer under the conditions described above.

In an alternative embodiment, a single mixer is used to obtain the desired high flow and low shear conditions. For example, a turbine or Ystral® mixer with high flow and low shear (such as the Ystral Jetstream) can be used at a typical running speed of approximately 15,000 rpm (+/−20%). It is appreciated that the Ystral Jetstream has a very low shear, and may be considered to be a non-shear mixer. This will result in the complete incorporation of the neutralising agent into the bigel without degradation of the carbopol gel matrix, resulting in a bigel which can have a thick gel matrix with a viscosity in excess of 400,000 cP (using the method of viscosity measurement specified in the Examples).

Thus, by “high flow and low shear mixing” we include the meaning of mixing under flow and shear conditions which are equivalent to those obtained using the Ystral Jetstream mixer at a running speed of 12,000 to 18,000 rpm.

It is preferred if the pH adjustment or neutralisation stage is conducted under vacuum to prevent the incorporation of air into the product.

It is appreciated that the pH adjustment or neutralisation stage can be conducted as one, two, three or more distinct stages. Thus, in the production method detailed in the Examples, a first neutralisation step comprises the addition of sodium methylparaben solution, and second and third neutralisation steps comprise the addition of sodium hydroxide solutions, each of these stages was conducted using high flow and low shear mixing.

The relationship between pH and the viscosity of a composition containing carbomers (e.g. Carbopols®) are known to the skilled person, as is the relationship between the concentration of the carbomer, its pH and viscosity.

For example, Noveon™ Pharmaceutical Polymers Bulletin No. 11 (2002) “Thickening Properties”, (http://www.pharma.noveoninc.com/literature/bulletin/epb11.pdf) describes the relationship between the pH and viscosity of a number of Carbopol® polymers at various concentrations. FIGS. 11.1.1 to 11.1.7 and Table 11.2.1 show the effect of pH on viscosity of 0.2%, 0.5% and 1.0% solutions of Carbopol 940NF, Carbopol 934 NF and Carbopol 941 NF, neutralised with 10% NaOH or 50% triethanolamine; FIGS. 11.2.1 to 11.2.3 show the thickening efficiency of various Carbopol® polymers at different concentrations; and FIGS. 11.3.1 to 11.3.3 show the effect of salts such as sodium chloride on viscosity. The viscosities given in this Bulletin are Brookfield Viscosities measured at 20 rpm and, although the temperature is not specified, the product specifications for Noveon™ Carbopols® specify a temperature of 25° C. In any event, FIG. 11.6.1 shows that the viscosity of Carbopol® polymers is relatively temperature-insensitive, decreasing slightly at higher temperatures. The entire disclosure of Noveon™ Pharmaceutical Polymers Bulletin No. 11 relating to the thickening properties of Carbopol® polymers is incorporated herein by reference.

Based on the information contained in Noveon™ Pharmaceutical Polymers Bulletin No. 11, together with his routine skills and abilities, the skilled person can readily determine suitable combinations of gelling agent (both specific type and concentration) and pH adjusting agent (type and concentration) required to obtain a bigel of a desired final pH and desired final viscosity suitable for its intended use.

The viscosity to be achieved as a result of the pH adjustment step is dependent upon the intended use of the bigel. For example, “shower gels” typically have a viscosity within the range 1,000-20,000 cP (1-20 Pa·s), lotions typically have a viscosity within the range 1,000-30,000 cP (1-30 Pa·s), while creams and ointments have a higher viscosity above 30,000 cP (30 Pa·s), preferably above 80,000 cP (80 Pa·s). Suitable creams or ointments typically have a viscosity within the range of 30,000-150,000 cP (30-150 Pa·s), and more preferably between 80,000-140,000 cP (80-140 Pa·s) or 90,000-120,000 cP (90-120 Pa·s) when measured using a Lamy VRM-08 viscometer using an appropriate MS DIN module at a temperature of 23° C. and at a shear stress of 0.8 s⁻¹.

Thus, step (d) of adjusting the pH of the bigel to obtain a bigel of a desired viscosity, may be considered to include adjusting the pH of the mixture to obtain a bigel of a suitable viscosity for use as a cream or ointment as is well known in the art.

The viscosity of a bigel cream made as described in the Examples with a desired feel and viscosity was measured using a Lamy VRM-08 viscometer using an MS DIN 1.3 module at a temperature of 23° C. and at a shear stress of 0.8 s⁻¹, giving a measurement after 30 seconds of 117,000 mPa·s (cP)±5,000. Accordingly, in an embodiment, preferred viscosities of a bigel cream produced according to the present invention are 95,000-140,000 mPa·s., and more specifically 112,000-122,000 mPa·s., when measured under these conditions.

Similarly, the viscosity of four batches of bigel cream made as described in the Example, below, with a desired feel and viscosity was measured using a Brookfield mobile B viscometer at 0.5 rpm at a temperature of 25° C. giving a measurement of 340,000 to 380,000 mPa·s (cP). Accordingly, in an embodiment, preferred viscosities of a bigel cream produced according to the present invention are 300,000-420,000 mPa·s., and more specifically 340,000-380,000 mPa·s., when measured under these conditions.

It is also appreciated that the final pH of the bigel depends upon its intended use. For example, human skin has a pH of about 6 to 6.5, the vagina has a pH of about 3.5 to 4, and the eye has a pH about 7, and the mouth has a pH of about 6.5. It is clearly preferred if the bigel has a pH which is close to the physiological pH of the intended target area. Similarly, it is preferred if the bigel has a pH which is compatible with the activity of a drug or other active ingredient present in the bigel. Since Carbopol® polymers tend to reach a flat viscosity plateau at between pH 5 and 10, it is generally possible to obtain a bigel of a desired viscosity, and having a pH suitable for its intended use.

Thus, it is within the routine abilities of the skilled person to determine the type and amount of gelling agents in the aqueous phase to obtain a bigel of desired viscosity and having the desired pH.

It is appreciated that it is not always necessary to measure the viscosity of a bigel in order to make a bigel having a desired viscosity. For example, whether a bigel has a viscosity suitable for use as a lotion or cream or ointment, can be determined by feel. In addition, once the components and conditions for making a bigel having a desired viscosity have been determined, it is possible to recreate a bigel having that specific viscosity by following the same procedures.

The pH adjustment step may include adjusting the pH to between about pH 3-3.5, or to pH 3.5-4, or to pH 4-4.5, or to pH 4.5-5, or to pH 5-5.5, or to pH 5.5-6, or to pH 6-6.5, or to pH 6.5-7, or to pH 7-7.5, or to pH 7.5-8, or to pH 8-8.5, or to between about pH 8.5-9.

Suitable bigels have been formed with a pH of between 4.9 and 6, and for some applications, the ideal pH of the bigel is around pH 5.

For a bigel formulation intended for human topical use, the pH adjustment step may be a neutralisation step. By “neutralisation” we include the meaning of bringing the pH to about pH 7. We also include the meaning of bringing the pH of the bigel towards pH 7, without necessarily reaching this value.

Ethylcellulose-based carbomers are strongly acidic, and the pH of the bigel prior to the final pH adjustment/neutralisation step is a result of the percentage of carbomer present in the aqueous phase and can be calculated or measured by a person of skill in the art.

Typically, step (d) of adjusting the pH of the bigel to obtain a bigel of a desired viscosity comprises adding one or more pH adjusting-agents, sometimes referred to as neutralising agents. Suitable pH adjusting-agents include (solutions of) sodium methylparaben, sorbic acid, sodium hydroxide, amino methyl propanol, sodium EDTA, HCl, potassium hydroxide, ammonium hydroxide, and some water-soluble organic amines such as triethanolamine. If inorganic bases are used to adjust the pH of the solution, a stable water soluble gel is formed. If triethanolamine is used, the gel can tolerate high alcohol concentrations.

The choice of pH adjusting-agent may depend upon the nature of the aqueous phase. For example, if the aqueous phase was buffered, and if it is necessary to reduce the pH, an appropriate acid may need to be used, such as phosphoric acid.

Some pH adjusting-agents such as sodium methylparaben and sorbic acid may also be included in the bigel as a preservative. Thus, for example, the method may include adding sodium methylparaben to the bigel composition after mixing the oily and aqueous phases, to act as both as a preservative and as a pH adjusting agent. In an embodiment, sodium hydroxide may also be used if, following addition of the sodium methylparaben, the pH is still below 5 and the desired pH is higher. Alternatively, NaOH may be used as the sole a pH adjusting agent.

In an embodiment, the method also comprises adding one or more standard ingredients for gels, creams and ointments, including texture agents, antioxidants such as butylated hydroxyanisole (BHA) or butylated hydroxytoluene (BHT), dyes/colouring agents or fragrances. They can be added to the oleogel or the aqueous gel before mixing, or added to the bigel after the phases have been combined.

Other components that can be added include antimicrobial preservation agents such as sodium methylparaben, sodium propylparaben, sorbic acid and dimethyloldimethylhydantoin (Glydant XL 1000). Preferably, these are included at between about 0.01 to 0.5%, by weight of the final bigel composition, and may suitably be employed at levels of about 0.01 to 0.05%, 0.05 to 0.1%, 0.1 to 0.2%, 0.2 to 0.3%, 0.3 to 0.4% and 0.4 to 0.5% by weight.

When the bigel produced by the methods of the present invention is intended for use as a pharmaceutical composition, the bigel comprises at least one active pharmaceutical ingredient (API). Active ingredients can be added to either or both of the oleogel and the aqueous gel.

The bigel may also include an active ingredient in the oily phase and a second active ingredient in the aqueous phase, the two active ingredients being incompatible with each other. By “incompatible active ingredients” we include active ingredient which are capable of reacting together in a chemically or therapeutically undesirable manner. Insofar as the incompatible active ingredients are mainly soluble, respectively, in the oily phase or the aqueous phase of the bigel, adverse reactions between the incompatible active ingredients before their administration can be avoided.

It is appreciated that the viscosity of the bigel may be adjusted to an appropriate value for the intended route of administration of the bigel, typically by adapting the proportions of gelling agents in the aqueous gel phase, or, less preferably, in the oleogel phase.

For example, given its biodegradable nature, the bigel composition can be administered as an injection. It is thus possible to inject allergens or vaccines which are known to be injected in difficult conditions and/or effective only under special administration conditions e.g. in a special solvent that improves antibody formation, such as the incomplete Freund's adjuvant (a mixture of an internal aqueous phase dispersed in an external oily phase consisting of mannide monooleate (Arlacel A)). The incomplete Freund's adjuvant, however, causes serious inflammatory side effects at the site injection, because it causes a chronic inflammatory response that may be severe and painful depending on the site as well as the quantity and quality of adjuvant injected. The inflammatory response may result in formation of chronic granulomas, sterile abscesses, and/or ulcerating tissue necrosis. In contrast, the bigel may have the same beneficial properties in aiding the immune response and stimulating antibody formation, but do not have such inflammatory side effects.

For certain intended uses, in particular for the purpose of a topical administration, the bigel can comprise the same active ingredient in the oleogel and the aqueous gel. This is because the inventors have found the profile for release of the active ingredient into the skin from a bigel is considerably better than that obtained with a composition based on only one type of gel, oily or aqueous, for a given volume of composition applied and a given concentration of active ingredient relative to the total weight of the composition. This is particularly advantageous for using the bigel as an active ingredient reservoir in a transdermal release system. On the skin, the active ingredient(s) dissolved in the aqueous gel phase is rapidly taken up by the epidermal layers, which allows the percutaneous passage to start quickly. The active ingredient(s) in the oleogel phase must firstly, as a function of its oil/water partition coefficient, pass into the aqueous gel phase in order to pass into the epidermal layers: this fraction in the oleogel will thus be released with a greater delay, after the active ingredient(s) dissolved in the aqueous gel phase has been released.

Suitable active ingredients that can be included in a bigel for pharmaceutical use include allergens and vaccines, substances such as hormones which require sustained release while at the same time maintaining good tolerance, dermatologically active ingredients, antimicrobial agents, cancer chemotherapeutic agents, anti-inflammatory agents and wound repair agents, as well as any of the agents listed below.

In an preferred embodiment, the bigel may comprises one or more hormones such as β-oestradiol, progesterone and testosterone.

Suitably, the oestradiol (or other oestrogen), progesterone and/or testosterone can be present in a bigel at between 0.5 and 2.5% by weight, and more preferably at between 0.5 and 1.5% by weight, relative to the total weight of the bigel. In an embodiment, a 1% formulation is preferred.

Typically, oestradiol is included in the oleogel, preferably in a proportion of between 0.1 and about 5% by weight, relative to the total weight of the oleogel. Typically, progesterone is included in the aqueous phase, preferably in a proportion of between about 1 and about 6% by weight, relative to the total weight of the aqueous gel. Testosterone may be included in the oleogel and/or in the aqueous gel. Preferably the proportion of testosterone in the oleogel is between about 0.1 and 6% by weight, relative to the total weight of the oleogel, and the proportion of testosterone in the aqueous gel is between about 1 and 10% by weight, relative to the total weight of the aqueous gel.

Similarly at least one corticoid or corticoid derivative may be present in the oleogel and the aqueous gel, the proportion of the corticoid derivative in the oleogel being between about 0.001 and about 3% by weight, relative to the total weight of the oily phase, and the proportion of the corticoid derivative in the aqueous gel being between about 0.001 and about 3% by weight, relative to the total weight of the aqueous phase. By “corticoid derivative” we include the meaning of any structural, natural or synthetic derivative of the corticoids, including cortisone, 11-oxycorticosteroids, in particular hydroxycortisone.

Suitable antimicrobial agents include antibacterial agents, antifungal agents and antiviral agents, as is well known to the person of skill in the art. Antibacterial agents include, for example natural and synthetic penicillins and cephalosporins, sulphonamides, erythromycin, kanamycin, azithromycin, clarithromycin, roxithromycin, tetracycline, oxytetracycline, chloramphenicol, rifampicin and including gentamicin, ampicillin, benzypenicillin, benethamine penicillin, benzathine phenethicillin, phenoxy-methyl penicillin, procaine penicillin, cloxacillin, flucloxacillin, methicillin sodium, amoxicillin, bacampicillin hydrochloride, ciclacillin, mezlocillin, pivampicillin, talampicillin hydrochloride, carfecillin sodium, piperacillin, ticarcillin, mecillinam, pirmecillinan, cefaclor, cefadroxil, cefotaxime, cefoxitin, cefsulodin sodium, ceftazidime, ceftizoxime, cefuroxime, cephalexin, cephalothin, cephamandole, cephazolin, cephradine, cefpirome, latamoxef disodium, aztreonam, glycylcyclines, chlortetracycline hydrochloride, clomocycline sodium, demeclocydine hydrochloride, doxycycline, lymecycline, minocycline, oxytetracycline, amikacin, framycetin sulphate, neomycin sulphate, netilmicin, tobramycin, colistin, sodium fusidate, fupirocin, polymyxin B sulphate, spectinomycin, vancomycin, teicoplanin, calcium sulphaloxate, sulphametopyrazine, sulphadiazine, sulphadimidine, sulphaguanidine, sulphaurea, capreomycin, metronidazole, tinidazole, cinoxacin, ciprofloxacin, norfloxacin, nitrofurantoin, hexamine, streptomycin, carbenicillin, colistimethate, polymyxin B, furazolidone, nalidixic acid, trimethoprim-sulphamethoxazole, clindamycin, lincomycin, cycloserine, isoniazid, ethambutol, ethionamide, pyrazinamide, meropenem and imipenem and the like; anti-fungal agents include, for example miconazole, ketoconazole, itraconazole, fluconazole, amphotericin, flucytosine, griseofulvin, natamycin, nystatin, and the like; and anti-viral agents include agents such as acyclovir, AZT, ddI, amantadine hydrochloride, inosine pranobex, vidarabine, and the like.

Suitable cancer chemotherapeutic agents include: alkylating agents including nitrogen mustards such as mechlorethamine (HN₂), cyclophosphamide, ifosfamide, melphalan (L-sarcolysin) and chlorambucil; ethylenimines and methylmelamines such as hexamethylmelamine, thiotepa; alkyl sulphonates such as busulphan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

Further active agents which can be used with the present invention include all drugs which can be delivered onto or through the skin for either a local or systemic effect. These compounds include agents in all of the major therapeutic areas, including, but not limited to, ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha-two-adrenergic agonists, analgesics, antipyretics and anti-inflammatory agents, androgens, local and general anesthetics, antiaddictive agents, antiandrogens, antiarrhythmic agents, antiasthmatic agents, anticholinergic agents, anticholinesterase agents, anticoagulants, antidiabetic agents, antidiarrhoeal agents, antidiuretic, antiemetic and prokinetic agents, antiepileptic agents, antioestrogens, antifungal agents, antihypertensive agents, antimicrobial agents, antimigraine agents, antimuscarinic agents, antineoplastic agents, antiparasitic agents, antiParkinson's agents, antiplatelet agents, antiprogestins, antithyroid agents, antitussives, antiviral agents, atypical antidepressants, azaspirodecanediones, barbiturates, benzodiazepines, benzothiadiazides, beta-adrenergic agonists, beta-adrenergic antagonists, selective beta-1-adrenergic antagonists, selective beta-2-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, hematopoietic agents, histamines, histamine antagonists, 5-hydroxytryptamine antagonists, drugs for the treatment of hyperlipoproteinemia, hypnotics and sedatives, immunosuppressive agents, laxatives, methylxanthines, monoamine oxidase inhibitors, neuromuscular blocking agents, organic nitrates; pancreatic enzymes, phenothiazines, progestins, prostaglandins, agents for the treatment of psychiatric disorders, retinoids, sodium channel blockers, agents for spasticity and acute muscle spasms, succinimides, thioxanthines, thrombolytic agents, thyroid agents, tricyclic antidepressants, inhibitors of tubular transport of organic compounds, drugs affecting uterine motility, vasodilators, vitamins and the like.

Representative drugs include, for example, bepridil, diltiazem, felodipine, isradipine, nicardipine, nifedipine, nimodipine, nitredipine, verapamil, dobutamine, isoproterenol, carterolol, labetalol, levobunolol, nadolol, penbutolol, pindolol, propranolol, sotalol, timolol, acebutolol, atenolol, betaxolol, esmolol, metoprolol, albuterol, bitolterol, isoetharine, metaproterenol, pirbuterol, ritodrine, terbutaline, alclometasone, aldosterone, amcinonide, beclomethasone, dipropionate, betamethasone, clobetasol, clocortolone, cortisol, cortisone, corticosterone, desonide, desoximetasone, 11-desoxycorticosterone, 11-desoxycortisol, dexamethasone, diflorasone, fludrocortisone, flunisolide, fluocinolone, fluocinonide, fluorometholone, flurandrenolide, halcinonide, hydrocortisone, medrysone, 6α-methylprednisolone, mometasone, paramethasone, prednisolone, prednisone, tetrahydrocortisol, triamcinolone, benoxinate, benzocaine, bupivacaine, chloroprocaine, cocaine, dibucaine, dyclonine, etidocaine, lidocaine, mepivacaine, pramoxine, prilocaine, procaine, proparacaine, tetracaine, alfentanil, chloroform, clonidine, cyclopropane, desflurane, diethyl ether, droperidol, enflurane, etomidate, halothane, isoflurane, ketamine hydrochloride, meperidine, methohexital, methoxyflurane, morphine, propofol, sevoflurane, thiamylal, thiopental, acetaminophen, allopurinol, apazone, aspirin, auranofin, aurothioglucose, colchicine, diclofenac, diflunisal, etodolac, fenoprofen, flurbiprofen, gold sodium thiomalate, ibuprofen, indomethacin, ketoprofen, meclofenamate, mefenamic acid, mesalamine, methyl salicylate, nabumetone, naproxen, oxyphenbutazone, phenacetin, phenylbutazone, piroxicam, salicylamide, salicylate, salicylic acid, salsalate, sulphasalazine, sulindac, tolmetin, acetophenazine, chlorpromazine, fluphenazine, mesoridazine, perphenazine, thioridazine, trifluoroperazine, triflupromazine, diisopyramide, encainide, flecainide, indecainide, mexiletine, moricizine, phenyloin, procainamide, propafenone, quinidine, tocainide, cisapride, domperidone, dronabinol, haloperidol, metoclopramide, nabilone, proclorperazine, promethazine, thiethylperazine, trimethobenzamide, buprenorphine, butorphanol, dezocine, diphenoxylate, drocode, hydrocodone, hydromorphone, levallorphan, levorphanol, loperamide, meptazinol, methadone, nalbuphine, nalmefene, nalorphine, naloxone, naltrexone, oxybutynin, pentazocine, isosorbide dinitrate, nitroglycerin, theophylline, phenylephrine, ephidrine, pilocarpine, furosemide, tetracycline, chlorpheniramine, ketorolac, bromocriptine, guanabenz, prazosin, doxazocin, and flufenamic acid.

Other representative drugs include benzodiazepines, such as alprazolam, brotizolam, chlordiazepoxide, clobazam, clonazepam, clorazepate, demoxepam, diazepam, flumazenil, flurazepam, halazepam, lorazepam, midazolam, nitrazepam, nordazepam, oxazepam, prazepam, quazepam, temazepam, triazolam, and the like; an antimuscarinic agent such as anisotropine, atropine, clidinium, cyclopentolate, dicyclomine, flavoxate, glycopyrrolate, hexocyclium, homatropine, ipratropium, isopropamide, mepenzolate, methantheline, oxyphencyclimine, pirenzepine, propantheline, scopolamine, telenzepine, tridihexethyl, tropicamide, and the like; an estrogen such as chlorotrianisene, diethylstilbestrol, methyl estradiol, estrone, estrone sodium sulphate, estropipate, mestranol, quinestrol, sodium equilin sulphate, 17β-estradiol (or estradiol), semi-synthetic estrogen derivatives such as the esters of natural estrogen, such as estradiol-17β-enanthate, estradiol-17β-valerate, estradiol-3-benzoate, estradiol-17β-undecenoate, estradiol 16,17-hemisuccinate or estradiol-17β-cypionate, and the 17-alkylated estrogens, such as ethinyl estradiol, ethinyl estradiol-3-isopropylsulphonate, and the like; an androgen such as danazol, fluoxymesterone, methandrostenolone, methyltestosterone, nandrolone decanoate, nandrolone phenpropionate, oxandrolone, oxymetholone, stanozolol, testolactone, testosterone, testosterone cypionate, testosterone enanthate, testosterone propionate, and the like; or a progestin such as ethynodiol diacetate, gestodene, hydroxyprogesterone caproate, levonorgestrel, medroxyprogesterone acetate, megestrol acetate, norethindrone, norethindrone acetate, norethynodrel, norgestrel, progesterone, and the like.

Particularly preferred are contraceptive hormones progestogen, norethisterone, dydrogesterone, levonorgestrel, medroxyprogesterone, norgestrel, tinolone, dydrogesterone, desogestrone, drospirenone, gestodene, levonorgestrel, norelgestromin and norethisterone.

In embodiments where the-active drugs produce a local effect, the agents include, but are not limited to (in addition to local agents listed above), antiviral agents (e.g., acyclovir and idoxuridine, etc.), antifungal agents (e.g., amphotericin B, clotrimazole, nystatin, ketoconazole, miconazole, butoconazole, haloprogin, etc.), antibiotic agents (penicillins, cephalosporins erythromycin, tetracycline, clindamycin, aminoglycosides, chloramphenicol, polymixin b, bacitracin, neomycin, gentamycin etc.), antiseptics (e.g., povidone-iodine, S methylbenzethonium chloride, etc.), antiparasitics (e.g., lindane, anthralin, etc.) analgesic agents (e.g., methylsalicylate, salicylic acid, dyclonine, aloe vera etc.), local anesthetics (e.g., benzocaine, lidocaine, xylocaine, butamben picrate, etc.), anti-inflammatory agents (e.g., steroidal compounds such as dexamethasone, betamethasone, prednisone, prednisolone, triamcinolone, hydrocortisone, alclometasone, amcinonide, diflorasone, etc. as well as non-steroidal anti-inflammatories), anti-itch and irritation-reducing compounds (e.g., antihistamines such as diphenhydramine and psoriasis treatments); burn relief compounds (e.g., o-amino-p-toluenesulphonamide, monoacetate, etc.); depigmenting agents (e.g., monobenzone); and hormonal agents (e.g., oestriol).

The compounds that can be included in a bigel, including the compounds listed above, are meant to include all pharmaceutically acceptable salts and conjugates.

Other topically-active compounds are listed in Remington's Pharmaceutical Sciences, 17th Ed., Merck Publishing Co., Easton, Pa. (1985), pages 773-791 and pages 1054-1058, incorporated herein by reference.

It is also appreciated that the bigel may be intended to deliver an active agent to the intestines, perhaps coated by a capsule as is well known in the art. In this embodiment, the bigel may contain live microorganisms for timed release to the bowel.

For intended topical administration, at least one of the oily sunscreen filters described above may be included provided that it is not incompatible with any active pharmaceutical ingredient present in the oleogel, i.e. there is no chemically or therapeutically undesirable reaction between the oily sunscreen filter and the pharmaceutical ingredient.

Typically, the active ingredients are provided in, or added to, the oleogel and/or the aqueous gel in steps (a) and (b) of the method described above, before the two phases are mixed together and subsequently neutralised. This is the case, in particular, when at least two mutually incompatible active ingredients are added, one to the oleogel and the other to the aqueous gel. It is appreciated that it is possible to add an active ingredient to the bigel, with stirring, so as to distribute the active ingredient in both the oleogel and the aqueous gel.

A person skilled in the art will know to take into account the characteristics of the active ingredient used, adapting the operating conditions of the process, in particular the temperature, so as possibly not to adversely affect the properties of the active ingredient.

Thus in a preferred embodiment, the method further comprises packaging and presenting the bigel for use in medicine.

It is appreciated that the certain of the components listed above for pharmaceutical uses may be included in a bigel for cosmetic purposes. Similarly, certain of the components listed below for cosmetic uses may also be included in a bigel intended for pharmaceutical purposes.

For the preparation of a bigel for cosmetic purposes, the method may comprise adding at least one cosmetically active ingredient. The cosmetically active ingredient can be any ingredient which gives a cosmetic effect, i.e. an ingredient which, when placed in contact with the various external parts of the human body (epidermis, pilous and hair system, nails, lips and outer genital organs) or with the teeth or oral mucosae, by means of the composition, makes it possible, exclusively or mainly, to cleanse them, to fragrance them, to modify their appearance and/or to correct body odours and/or to protect them or keep them in good condition.

The cosmetically active ingredient may be a moisturiser, sunscreen, anti-free-radical agent or skin regenerator which are soluble in either the oleogel or the aqueous gel phases. Advantageously, given the problems posed by body-care oils in cosmetics, such as bath oils, as explained above, the cosmetically active ingredient may be chosen from the body-care oils, and more particularly from bath oils such as palm oil, sesame oil or argan oil. It is therefore appreciated that the oily agent may also constitute a cosmetically active ingredient. Depending on the type of cosmetically active ingredient used, a person skilled in the art will know how to determine its appropriate proportion by weight, relative to the total weight of the bigel for cosmetic uses.

Further active ingredients that may advantageously be included in a bigel formulated for cosmetic use include personal care actives which can be beneficial to the skin and/or hair. Suitable active ingredients may be selected from antimicrobial and antifungal actives, vitamins, anti-acne actives; anti-wrinkle, anti-skin atrophy and skin repair actives; skin barrier repair actives; non-steroidal cosmetic soothing actives; artificial tanning agents and accelerators; skin lightening actives; sunscreen actives; sebum stimulators; sebum inhibitors; anti-oxidants; protease inhibitors; skin tightening agents; anti-itch ingredients; hair growth inhibitors; 5-alpha reductase inhibitors; desquamating enzyme enhancers; anti-glycation agents; anti-perspirants, topical anesthetics, or mixtures thereof; and the like. These active agents may be selected from water soluble active agents, oil soluble active agents, pharmaceutically-acceptable salts and mixtures thereof.

The safe and effective amount of active agent ingredients will depend upon the specific active agent, its ability to penetrate the skin, the age, health condition, and skin condition of the expected user, and other similar factors. Preferably the bigel will comprise from about 0.01% to about 10%, more preferably from about 0.05% to about 5%, even more preferably 0.1% to about 5%, and most preferably 0.1% to about 2%, by weight of the active agent component.

Anti-acne actives can be effective in treating acne vulgaris, a chronic disorder of the pilosebaceous follicles. Examples of useful anti-acne actives include the keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulphur-containing D and L amino acids and their derivatives and salts, particularly their N-acetyl derivatives, mixtures thereof and the like.

Antimicrobial and antifungal actives can be effective to prevent the proliferation and growth of bacteria and fungi. Examples of antimicrobial and antifungal actives include θ-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, triclosan; triclocarban; and mixtures thereof and the like.

Anti-wrinkle, anti-skin atrophy and skin repair actives can be effective in replenishing or rejuvenating the epidermal layer. These actives generally provide these desirable skin care benefits by promoting or maintaining the natural process of desquamation. Examples of anti-wrinkle and anti-skin atrophy actives include vitamins, minerals, and skin nutrients such as milk, vitamins A, E, and K; vitamin alkyl esters, including vitamin C alkyl esters; magnesium, calcium, copper, zinc and other metallic components; retinoic acid and its derivatives (e.g., cis and trans); retinal; retinol; retinyl esters such as retinyl acetate, retinyl palmitate, and retinyl propionate; vitamin B₃ compounds (such as niacinamide and nicotinic acid), alpha hydroxy acids, beta hydroxy acids, e.g. salicylic acid and derivatives thereof (such as 5-octanoyl salicylic acid, heptyloxy 4 salicylic acid, and 4-methoxy salicylic acid); mixtures thereof and the like.

Skin barrier repair actives are those skin care actives which can help repair and replenish the natural moisture barrier function of the epidermis. Examples of skin barrier repair actives include lipids such as cholesterol, ceramides, sucrose esters and pseudo-ceramides, ascorbic acid; biotin; biotin esters; phospholipids, mixtures thereof, and the like.

Non-steroidal cosmetic soothing actives can be effective in preventing or treating inflammation of the skin. The soothing active enhances the skin appearance benefits of the present invention, e.g., such agents contribute to a more uniform and acceptable skin tone or colour. Examples of cosmetic soothing agents include the following categories: propionic acid derivatives; acetic acid derivatives; fenamic acid derivatives; mixtures thereof and the like.

Artificial tanning actives can help in simulating a natural suntan by increasing melanin in the skin or by producing the appearance of increased melanin in the skin. Examples of artificial tanning agents and accelerators include dihydroxyacetone; tyrosine; tyrosine esters such as ethyl tyrosinate and glucose tyrosinate; mixtures thereof, and the like.

Skin lightening actives can actually decrease the amount of melanin in the skin or provide such an effect by other mechanisms. Examples of skin lightening actives useful herein include aloe extract, alpha-glyceryl-L-ascorbic acid, aminotyroxine, ammonium lactate, glycolic acid, hydroquinone, 4 hydroxyanisole, mixtures thereof, and the like.

Also useful herein are sunscreen actives. A wide variety of sunscreen agents are known in the art. Examples of sunscreens which may be useful in the compositions of the present invention are those selected from the group consisting of octyl methoxyl cinnamate (Parsol MCX) and butyl methoxy benzoylmethane (Parsol 1789), 2-ethylhexyl p-methoxycinnamate, 2-ethylhexyl N,N-dimethyl-p-aminobenzoate, p-aminobenzoic acid, 2-phenylbenzimidazole-5-sulphonic acid, oxybenzone, mixtures thereof, and the like.

Sebum stimulators can increase the production of sebum by the sebaceous glands. Examples of sebum stimulating actives include bryonolic acid, dehydroetiandrosterone (DHEA), orizanol, mixtures thereof, and the like.

Sebum inhibitors can decrease the production of sebum by the sebaceous glands. Examples of useful sebum inhibiting actives include aluminum hydroxy chloride, corticosteroids, dehydroacetic acid and its salts, dichlorophenyl imidazoldioxolan (available from Elubiol), mixtures thereof, and the like.

Protease inhibitors may also be useful as actives in a bigel formulated for cosmetic use. Protease inhibitors can be divided into two general classes: the proteinases and the peptidases. Proteinases act on specific interior peptide bonds of proteins and peptidases act on peptide bonds adjacent to a free amino or carboxyl group on the end of a protein and thus cleave the protein from the outside. The protease inhibitors suitable for use in the present invention include, but are not limited to, proteinases such as serine proteases, metalloproteases, cysteine proteases, and aspartyl protease, and peptidases, such as carboxypeptidases, dipeptidases and aminopeptidases, mixtures thereof and the like.

Other useful as active ingredients in the present invention are skin tightening agents. Examples of skin tightening agents which may be useful in the compositions of the present invention include monomers which can bind a polymer to the skin such as terpolymers of vinylpyrrolidone, (meth)acrylic acid and a hydrophobic monomer comprised of long chain alkyl (meth)acrylates, mixtures thereof, and the like.

Suitable examples of anti-itch ingredients which may be included in bigel compositions include hydrocortisone, methdilizine and trimeprazineare, mixtures thereof, and the like.

Examples of hair growth inhibitors which may usefully be included in the bigels of the present invention include 17 θ-oestradiol, anti angiogenic steroids, curcuma extract, cycloxygenase inhibitors, evening primrose oil, linoleic acid and the like.

Suitable 5-alpha reductase inhibitors include ethynylestradiol, genistine, and mixtures thereof, and the like.

Examples of desquamating enzyme enhancers which may optionally be included in the compositions of the present invention include alanine, aspartic acid, N methyl serine, serine, trimethyl glycine, mixtures thereof, and the like.

An example of an anti-glycation agent which may optionally be included in the bigel compositions of the present invention is Amadorine (available from Barnet Products Distributor), and the like.

Thus in another preferred embodiment, the method further comprises packaging and presenting the bigel for use as a cosmetic.

Suitable equipment for measuring the pH of a bigel composition comprises a WTW pH 340i pH-meter with a Sentix HW electrode. After calibration of the pH meter, three readings are taken and an average value calculated.

It is appreciated that alternative embodiments of the method of producing a bigel described in this first aspect of the invention may comprise an alternative step (d) of subsequently adjusting the pH of the bigel under high flow and low shear mixing to obtain a bigel having a rheological parameter (other than viscosity) at a desired level.

Suitable rheological parameters of a bigel other than viscosity include cohesion, adhesion and consistency index. Methods for determining cohesion, adhesion and consistency index of a bigel include the use of a Type TEC 025 texturometer with a sensor force of 20N and with a stainless steel hemispherical probe at 23° C.

Preferred cohesion values of a bigel measured under these conditions range between 0.8 to 0.95.

Preferred adhesion values of a bigel measured under these conditions range between 0.5 to 0.95 mJ.

Preferred consistency index values of a bigel measured under these conditions range between 2.5 to 6.5×10⁻² N/s⁻¹.

Thus, step (d) may be considered to include adjusting the pH of the bigel under high flow and low shear mixing to obtain a bigel having at least one rheological parameter (e.g. selected from viscosity, cohesion, adhesion and consistency index) at a desired level. Suitably, this step may comprise adjusting the pH of the bigel to obtain a bigel suitable for use as lotion or cream or ointment, as is well known in the art.

The invention includes a bigel obtainable by the methods of the first aspect of the invention.

The invention also includes a bigel obtained by the methods of the first aspect of the invention.

A second aspect of the invention provides a composition comprising:
at least one active agent selected from steroid hormones, glucosteroids and antimicrobial agents, each at 0.05-5% by weight;
a carbomer at 0.4-2% by weight;
caprylic/capric triglycerides at 10-30% by weight;
propylene glycol laurate and ethylcellulose and propylene glycol isostearate at 1-10% by weight, and
water.

It is appreciated that the each of the active agents may, independently, be at between 0.05-0.1% by weight in the composition, or about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or about 2.5% by weight in the composition. Alternatively, the active agents may be at between 2.5-3%, or 3-4% or at between 4-5% by weight in the composition. Preferably each of the active agents is, independently, present in the composition at between 0.5-2% by weight. It is appreciated that the dose of the active agent(s) to be administered is preferably determined by a physician, and hence the proportion by weight of the active ingredient will depend upon the expected quantity of the composition in each dose as well as upon the efficacy of each active agent.

Preferably, the steroid hormone is selected from testosterone, progesterone and oestrodiol. In a preferred embodiment, the composition comprises both testosterone and progesterone, or both testosterone and oestradiol, or both progesterone and oestradiol.

Preferably, the glucosteroid is selected from cortisol, hydrocortisone, cortisone acetate, prednisone, prednisolone, methylprednisolone, dexamethasone, betamethasone, triamcinolone, beclomethasone, fludrocrotisone acetate, deoxycorticosterone acetate (DOCA) and aldosterone.

Suitable antimicrobial agents are listed above, and include antibacterial agents, antifungal agents and antiviral agents as is well known to the person of skill in the art.

It is appreciated that the composition may comprise propylene glycol laurate, ethylcellulose and propylene glycol isostearate, taken together, at about 1% by weight, or about 2%, or about 3%, or about 4%, or about 5%, or about 6%, or about 7%, or about 8%, or about 9%, or about 10%, by weight. Preferably, the propylene glycol laurate and ethylcellulose and propylene glycol isostearate, taken together, are present at 3-6% by weight.

Also preferably, the ethylcellulose, propylene glycol isostearate and propylene glycol laurate are present in the relative proportions discussed above with reference to the first aspect of the invention.

The composition of the second aspect of the invention may, optionally contain other components such as those described above with reference to the first aspect of the invention. Components that can be mentioned include any one or more of:
sorbic acid at 0.05-0.4% by weight;
sodium methylparaben at between 0.05 and 0.4% by weight;
sodium propylparaben at between 0.05 and 0.4% by weight; and
ethanol at between 1 and 10% by weight.
BHA at between 0.05-0.4% by weight; and
BHT at between 0.05-0.4% by weight.

In an embodiment, the composition is in the form of a lotion having a viscosity within the range 1,000-30,000 cP (1-30 Pa·s) measured using a Lamy VRM-08 with an MS DIN 1.9 module at 23° C., and preferably between 20-30,000 cP (20-30 Pa·s). In an alternative and more preferred embodiment, the composition may be in the form of a cream or ointment having a viscosity above 30,000 cP (30 Pa·s), preferably above 80,000 cP (80 Pa·s) measured using a Lamy VRM-08 with an MS DIN 1.3 module at 23° C. Suitable creams or ointments typically have a viscosity within the range of 30,000-150,000 cP (30-150 Pa·s), and more preferably between 80,000-140,000 cP (80-140 Pa·s), or between 90,000-120,000 cP (90-125 Pa·s), or between 112,000-122,000 cP (112-122 Pa·s), measured using the Lamy VRM-08 with an MS DIN 1.3 module at 23°.

Suitably, the composition has a pH of between about pH 3-3.5, or pH 3.5-4, or pH 4-4.5, or pH 4.5-5, or pH 5-5.5, or pH 5.5-6, or pH 6-6.5, or pH 6.5-7, or pH 7-7.5, or pH 7.5-8, or pH 8-8.5, or between about pH 8.5-9. As is appreciated by the skilled person, the desired pH is dependent upon the intended use of the composition. For example, compositions for topical administration preferably have an approximately neutral pH of between 5.5-8, while compositions for vaginal administration are generally more acidic, having a typical pH of between 3-5.

Preferably, the composition is not an emulsion. Therefore, in this embodiment, the composition does not require the presence of an emulsifying agent, such as those listed above.

More preferably, the composition is a bigel as defined above.

In a most preferred embodiment, the composition is made by the methods of the first aspect of the invention.

All of the documents referred to herein are incorporated herein, in their entirety, by reference.

The listing or discussion of a prior-published document in this specification should not necessarily be taken as an acknowledgement that the document is part of the state of the art or is common general knowledge.

The invention will now be described in more detail by reference to the following FIGURE and Examples. In the Examples, except where otherwise specified, the percentages indicated are percentages by weight.

FIG. 1: A flow-chart of the bigel manufacturing process used in Example 1.

### EXAMPLE 1

### Bigel Manufacturing Process

The following manufacturing process was conducted using a 50 L Olsa MPEF equipped with a planetary mixer (plough with scrapper) (0-60 rpm) and a disperser homogeniser (1500/3000 rpm) rotor/stator type, on the vessel bottom.

### Bigel Components

Placebo and 1% testosterone-containing batches were prepared from the following components:

TABLE 2
Formula for placebo batches

| Raw material | Percent quantity | Quantity for a 40 kg batch | |
|---|---|---|---|
| Carbopol 980 NF | 1.40% | 560.00 | g |
| Labrafac CC | 21.00% | 8,000.00 | g |
| Emulfree P | 4.00% | 1,600.00 | g |
| Ethanol 96% | 3.50% | 1,400.00 | g |
| Purified water | 69.55% | 25,888.00 | g |
| Na methylparaben | 0.25% | 100.00 | g |
| Sorbic acid | 0.13% | 52.00 | g |
| NaOH pellets EP | 0.17% | 68.00 | g |
| Total | 100.00% | 40,000.00 | g |

TABLE 3
Formula for 1% testosterone batch

| Raw material | Percent quantity | Quantity for a 40 kg batch | |
|---|---|---|---|
| Testosterone | 1.00% | 400.00 | g |
| Carbopol 980 NF | 1.40% | 560.00 | g |
| Labrafac CC | 20.00% | 8,000.00 | g |
| Emulfree P | 4.00% | 1,600.00 | g |
| Ethanol 96% | 3.50% | 1,400.00 | g |
| Purified water | 69.55% | 25,888.00 | g |
| Na methylparaben | 0.25% | 100.00 | g |
| Sorbic acid | 0.13% | 52.00 | g |
| NaOH pellets EP | 0.17% | 68.00 | g |
| Total | 100.00% | 40,000.00 | g |

### Description of Manufacturing Process

### 1. Preparation of the Aqueous Phase:

a) Dissolution of sorbic acid in water heated at 50° C. during 15 minutes in the Olsa with planetary mixer at 15 rpm and disperser at 1500 rpm (MIX A).

### b) Cooling at 25° C.-30° C.

c) Manual addition of Carbopol 980 in the Olsa with disperser at 1500 rpm at the beginning, then 3000 rpm until complete swelling of the Carbopol.
d) Mixing under vacuum for 1 h with planetary at 15 rpm and disperser at 3000 rpm (MIX B).

### 2. Preparation of the Oily Phase (in Parallel to the Phase 1.d):

a) Addition of testosterone in a 20 l vessel containing the Labrafac CC and mixing with a rotor stator mixer (Ultra-Turrax) during 5 min (MIX C).
b) Addition of ethanol (during 5 min) to the mixture with a Rayneri blade mixer (MIX D).
c) Addition of Emulfree P (during 10 min) to the mixture with a Rayneri blade mixer (MIX E).

### 3. Preparation of the Bigel:

a) Mixing of the 2 phases: the oily phase is incorporated slowly and regularly in 20 minutes into the aqueous phase in the Olsa, with disperser at 3000 rpm and planetary at 20 to 35 rpm.
b) After complete addition, the mixing is maintained 20 minutes under vacuum (MIX F).

### 4. Neutralization of the Bigel

a) During step 3b, the sodium methylparaben solution (SOL 1) is prepared, as well as NaOH solution (SOL 2) under agitation.
b) Incorporation by the upper Olsa funnel, of the paraben solution (SOL 1) in the Bigel under planetary mixing at 35 to 45 rpm and disperser mixing at 1500 rpm (slow addition during 25 min).
c) Incorporation by the upper Olsa funnel, of half of the NaOH solution (SOl 2) in the Bigel under planetary mixing at 45 to 55 rpm and disperser mixing at 1500 rpm (slow addition during 20 min).
d) After addition, blending during 5 minutes under vacuum.
e) Slow manual incorporation by the upper Olsa funnel of the remaining NaOH solution (SOL 2), with a 10 ml pipette, by fractions, under planetary mixing at 50 to 60 rpm only (during 20 min).
d) After addition, blending during 10 minutes under vacuum by planetary mixing at 55 rpm and disperser mixing at 1500 rpm.
NB: for the placebo formulation, the API is replaced by Labrafac CC, and the step of API dispersion is suppressed.

The neutralisation phase (incorporation of the sodium paraben solution and hydroxide sodium solution) is the most critical step during the manufacture of a bigel, and it was not simple to identify the conditions under which a homogenous viscous product can be obtained.

### Reasons for Process Failure

Before arriving at this successful manufacturing process for pilot-scale manufacture of a bigel, a number of factors were found to result in an unsuccessful process.

### Mixing Flow Too Low

The neutralisation was found to fail if the rate of mixing is too low i.e. if the planetary mixer alone is used for the incorporation of the neutralising agent. This has been demonstrated in scale up manufacturing batches: during gentle homogenisation splitting is observed at the end of the incorporation of NaOH solution. This occurs due to the incomplete incorporation of the neutralising agent, resulting in non-homogenous zones of thick and thin gel, which results in the splitting of the bigel.

### Mixing Shear Too High

If, during neutralisation, a high shear mixer alone is used to incorporate the neutralising agent into the gel, the carbopol gel matrix is broken down resulting in a reduction of the product viscosity. If the product viscosity is less than 300,000 cP (measured as specified below) the bigel begins to split.

In addition, the following parameters during the neutralisation step were tested and shown not to rectify an unsuccessful process: duration of incorporation of the solutions, incorporation order, volume of solution to use, influence of testosterone and influence of grade of Emulfree P.

### Reasons for Process Success

Mixing with High Flow and Low Shear

Use of the disperser (on its low speed setting; 1500 rpm) during and after the neutralisation steps. This is in addition to the use of the planetary mixer at 35-60 rpm. The additional flow generated by the planetary mixer in combination with the high shear mixer resulted in the incorporation of the neutralising agent into the gel without too much degradation of the carbopol gel matrix. This results in a gel with greater than 300,000 cP and a consistent, stable gel matrix.

Systematic use of the vacuum during the neutralization steps was also found to be beneficial

The bigel obtained following the manufacturing process described above was not split, and had the desired viscosity, correct pH (4.95) and a satisfactory and homogenous aspect.

Properties of a Bigel Made by this Process

During the manufacturing, in-process controls were recorded during the neutralization phase: appearance, pH and viscosity range of the bigel, and the results are shown in Tables 4 and 5.

The viscosity measurements were carried out using a Brookfield mobile B viscometer at 0.5 rpm at 25° C.

TABLE 4
Placebo Batch (ID1285)

| Process Stage | Appearance | Viscosity Range (cP) | pH |
|---|---|---|---|
| Bigel before neutralization | White creamy liquid | 88,000 to 100,000 | 2.67 |
| | gel | | |
| Bigel after addition of 100% Na | White, viscous, thick, | 520,000 to 560,000 | 3.94 |
| paraben solution (post 5 min. | homogenous gel | | |
| blend) | | | |
| Bigel after addition of 50% | White, viscous, thick, | 440,000 to 475,000 | 4.60 |
| NaOH solution (post 5 min. | homogenous gel | | |
| blend) | | | |
| Bigel after addition of 100% | White, viscous, thick, | 340,000 to 380,000 | 4.95 |
| NaOH solution (post 10 min. | homogenous gel | | |
| blend) | | | |

TABLE 5
Three 1% Testosterone Batches (ID1287; ID1303 and ID1307)

| Process Stage | Appearance | Viscosity Range (cP) | pH |
|---|---|---|---|
| Bigel before neutralization | Each batch: | 89,000 to 98,000 | 2.67 |
| | white creamy liquid gel | 88,000 to 94,000 | 2.64 |
| | | 83,000 to 90,000 | 2.69 |
| Bigel after addition of 100% Na | Each batch: | 490,000 to 570,000 | 3.98 |
| paraben solution (post 5 min. | white, viscous, thick, | 540,000 to 560,000 | 3.97 |
| blend) | homogenous gel | 530,000 to 570,000 | 3.93 |
| Bigel after addition of 50% | Each batch: | 420,000 to 460,000 | 4.60 |
| NaOH solution (post 5 min. | white, viscous, thick, | 430,000 to 470,000 | 4.61 |
| blend) | homogenous gel | 420,000 to 450,000 | 4.61 |
| Bigel after addition of 100% | Each batch: | 350,000 to 390,000 | 5.00 |
| NaOH solution (post 10 min. | white, viscous, thick, | 340,000 to 380,000 | 4.98 |
| blend) | homogenous gel | 350,000 to 380,000 | 4.99 |

These results show a good reproducibility of the results and robustness of the manufacturing process.

1. A method of producing a bigel, the method comprising:
(a) providing an oleogel comprising at least one oily agent gelled with at least one cellulose polymer;
(b) providing an aqueous gel comprising at least one component whose viscosity is pH dependent;
(c) mixing the oleogel and the aqueous gel together to form a bigel; and
(d) subsequently adjusting the viscosity of the bigel under high flow and low shear mixing to obtain a bigel of a desired viscosity.

2. A method according to claim 1 wherein the at least one cellulose polymer in the oleogel is selected from ethylcellulose, non-sodium carboxymethylcellulose, and mixtures thereof.

3. A method according to claim 1 wherein the at least one cellulose polymer in the oleogel is ethylcellulose.

4. A method according to claim 3 wherein the ethylcellulose is present in a proportion of between about 0.05% and 9% by weight of the bigel composition.

5. A method according to claim 1 wherein the oily agent in the oleogel is selected from mono-, di-, and triglycerides of synthetic, semi-synthetic and natural origin, and mixtures thereof.

6. A method according to claim 5 wherein the mono-, di-, and triglycerides of semi-synthetic origin comprise a mixture of polyoxy ethylenated oleic glycerides obtained by alcoholysis of natural plant oil.

7. A method according to claim 5 wherein the mono-, di-, and triglycerides of natural origin are selected from oils of plant origin, such as sweet almond oil, argan oil and palm oil.

8. A method according to claim 1 wherein the oily agent comprises a mixture of capric/caprylic triglycerides.

9. A method according to claim 1 wherein the oleogel further comprises one or both of propylene glycol isostearate and propylene glycol laurate, each present, independently, in a proportion of between about 0.1 and 5% by weight of the bigel composition.

10. A method according to claim 9 wherein the oleogel comprises ethylcellulose, propylene glycol isostearate and propylene glycol laurate and an oily agent.

11. A method according to claim 10 wherein the ethylcellulose, propylene glycol isostearate and propylene glycol laurate are present, in total, in a proportion of between about 2% and 6% by weight of the bigel composition.

12. A method according to claim 10 wherein the ethylcellulose, propylene glycol isostearate and propylene glycol laurate are present, in relative proportions of about 2-10:2-10:80-96, respectively.

13. A method according to claim 1 wherein step (a) of providing an oleogel, comprises making an oleogel as defined in any of claims 1-12.

14. A method according to claim 1 wherein the aqueous gel comprises at least one gelling agent whose viscosity varies and is pH dependent, selected from carbomers and sodium carboxymethylcellulose, and mixtures thereof.

15. A method according to claim 14 wherein the carbomer is selected from Carbopol® Ultrez 10, Carbopol® 71G, Carbopol® 910, Carbopol® 934, Carbopol® 934P, Carbopol® 940, Carbopol® 971P, Carbopol® 974, Carbopol® 974P, Carbopol® 980, Carbopol® 981, and Carbopol® 1342 NF.

16. A method according to claim 14 wherein the aqueous gel further comprises at least one gelling agent selected from poloxamers (Pluronics®), acacia, alginic acid, bentonite, ethylcellulose, hydroxyethylcellulose, hydroxypropylcellulo se, gelatin magnesium aluminum silicate (Veegum®), methylcellulose, polyvinyl alcohol, sodium alginate, tragacanth, and xanthan gum, and mixtures thereof.

17. A method according to claim 14 wherein the aqueous gel has a viscosity of between 100-1,000 cP (100-1,000 mPa·s) when measured using a Lamy VRM-08 with an MS DIN 1.9 module at 23° C.

18. A method according to claim 14 wherein the aqueous gel has a pH of between 2.5 and 4.

19. A method according to claim 1 wherein step (b) of providing an aqueous gel comprises making an aqueous gel comprising at least one gelling agent whose viscosity varies and is pH dependent.

20. A method according to claim 1 wherein the oleogel and aqueous gel are mixed in step (c) using high-shear mixing.

21. A method according to claim 1 wherein the at least one component whose viscosity is pH dependent comprises sodium carboxymethylcellulose, and the oleogel and aqueous gel are mixed in step (c) using low-shear mixing.

22. A method according to claim 1 wherein the adjustment step (d) comprises adding a pH adjusting agent selected from sodium hydroxide, potassium hydroxide, sodium methylparaben, sodium propylparaben, amino methyl propanol, sodium EDTA and triethanolamine.

23. A method according to claim 1 wherein adjusting step (d) comprises bringing the pH to between pH 4 and pH 6.

24. A method according to claim 1 wherein adjusting step (d) comprises bringing the pH to between pH 6 and pH 7.5.

25. A method according to claim 1 wherein the adjustment in step (d) raises the viscosity of the bigel to between 1,000-30,000 cP (1-30 Pa·s), and preferably between 20,000-30,000 cP (20-30 Pa·s), measured using a Lamy VRM-08 with an MS DIN 1.9 module at 23° C.

26. A method according to claim 1 wherein the adjustment in step (d) raises the viscosity of the bigel to between 30,000-150,000 cP (30-150 Pa·s), and preferably between 80,000-140,000 cP (80-140 Pa·s), measured using a Lamy VRM-08 with an MS DIN 1.3 module at 23° C.

27. A method according to claim 1 wherein the high flow and low shear mixing is conducted using a combination of a planetary mixer at about 40-45 rpm and a Silverson type mixer at about 1500 rpm.

28. A method according to claim 1 wherein the high flow and low shear mixing is conducted using a turbine or Ystral® mixer.

29. A method according to claim 1 wherein adjusting in step (d) is conducted under a vacuum.

30. A method according to claim 1 wherein an emulsifying agent is not added.

31. A method according to claim 1 further comprising the step of adding at least one preservative selected from sodium methylparaben, sodium propylparaben and sorbic acid.

32. A method according to claim 1 further comprising the step of adding at least one active pharmaceutical ingredient (API).

33. A method according claim 32 wherein the at least one API is selected from hormones, antimicrobial agents, allergens, vaccines, dermatologically active ingredients, cancer chemotherapeutic agents, anti-inflammatory agents, wound repair agents, analgesics, local anesthetics and anti-itch agents.

34. A method according claim 33 wherein the at least one API is a hormone selected from testosterone, progesterone and oestradiol.

35. A method according claim 34 wherein both testosterone and progesterone, or both testosterone and oestradiol, or both progesterone and oestradiol, are added.

36. A method according to claim 33 wherein the at least one anti-microbial agent is selected from antibacterial agents, antifungal agents and antiviral agents.

37. A method according to claim 1 further comprising the step of adding at least one further component selected from texture agents, antioxidants, dyes, or fragrances, moisturisers, sunscreens, anti-free-radical agents and skin regenerator agents.

38. A method according to claim 37 wherein the least one antioxidant is selected from butylated hydroxyanisole (BHA) and butylated hydroxytoluene (BHT).

39. A method according to claim 1 wherein the bigel is packaged and presented for use in medicine.

40. A method according to claim 1 wherein the bigel is packaged and presented for cosmetic use.

41. A bigel obtainable by the method of claim 1.

42. A method of producing a bigel from an oleogel and an aqueous gel, characterised in that after the oleogel and the aqueous gel have been mixed together, the pH of the mixture is adjusted under high flow and low shear stirring to obtain a bigel having a desired viscosity.

43. A composition comprising:
at least one active agent selected from steroid hormones, glucosteroids and antimicrobial agents, each at 0.1-5% by weight;
a carbomer at 0.4-2% by weight;
caprylic/capric triglycerides at 10-30% by weight;
propylene glycol laurate, ethylcellulose and propylene glycol isostearate, at 1-10% by weight; and
water.

44. A composition according to claim 43 wherein the steroid hormone is selected from testosterone, progesterone and oestradiol.

45. A composition according to claim 44 comprising testosterone and progesterone, testosterone and oestradiol, or progesterone and oestradiol.

46. A composition according to claim 43 wherein the glucosteroid is selected from cortisol, hydrocortisone, cortisone acetate, prednisone, prednisolone, methylprednisolone, dexamethasone, betamethasone, triamcinolone, beclomethasone, fludrocortisone acetate, deoxycorticosterone acetate and aldosterone.

47. A composition according to claim 43 wherein the antimicrobial agent is selected from antibacterial agents, antifungal agents and antiviral agents.

48. A composition according to claim 43 wherein the steroid hormone is a contraceptive hormone selected from progestogen, norethisterone, dydrogesterone, levonorgestrel, medroxyprogesterone, norgestrel, tinolone, dydrogesterone, desogestrone, drospirenone, gestodene, levonorgestrel, norelgestromin and norethisterone.

49. A composition according to claim 43 wherein each of the at least one active agents is present, independently, at about 0.5-2% by weight.

50. A composition according to claim 43 wherein the ethylcellulose, propylene glycol isostearate and propylene glycol laurate are present in relative proportions of about 2-10:2-10:80-96, respectively.

51. A composition according to claim 43 and having a pH of between 4-7.

52. A composition according to claim 43 further comprising any one or more of:
sorbic acid at between 0.05-0.4% by weight;
sodium methylparaben at between 0.05-0.4% by weight;
sodium propylparaben at between 0.05-0.4% by weight;
ethanol at between 1-10% by weight;
BHA at between 0.05-0.4% by weight; and
BHT at between 0.05-0.4% by weight.

53. A composition according to claim 43 that is a bigel.

54. A medicine comprising the composition according to claim 43.
